(19) United States
(12) Patent Application Publication
(10) Pub. No.: US 2023/0371498 A1
(43) Pub. Date: Nov. 23, 2023

(54) MICRO FROZEN BODY FOR LOADING DRUGS AND THE LOADING DEVICE THEREOF

(71) Applicant: Zhe Li, Shanghai (CN)

(72) Inventors: Zhe Li, Shanghai (CN); Xi Li, Shanghai (CN)

(21) Appl. No.: 17/748,041

(22) Filed: May 19, 2022

(51) Int. Cl. A01N 1/02; C12N 5/0775; A61P 17/02; A61K 38/18; A61K 35/12; A61K 9/00

(52) U.S. Cl. CPC A01N 1/0221; C12N 5/0662; A61P 17/02; A61K 38/18; A61K 35/12; A61K 9/0014

(57) ABSTRACT

A micro frozen body for loading drugs, the micro frozen body is formed of frozen liquid, the micro frozen body contains drugs, the micro frozen body releases drugs to the target tissue in the organism. The liquid is solidified by cryogenic freezing to form a solid-state micro frozen body, the drug loaded in micro freezing or the micro frozen body itself is formed of solidified drug solution, the micro frozen body can be made into a needle with certain hardness, the micro frozen body implements puncture, the low temperature of micro frozen body can reduce the aching feeling in the piercing process and constrict the capillary vessels on the site of puncture to reduce bleeding.

### BACKGROUND OF INVENTION

### 1. Field of the Invention

The present invention relates generally to a micro frozen body for loading drugs, and more particularly to a loading device thereof.

### 2. Description of Related Art

Skin indications such as pigmented spots and skin scars can be improved or treated by using some drugs; but it is difficult to supply drugs to subcutaneous tissue. In the past, the drugs were smeared on the skin and slowly absorbed into the skin, which is inefficient. In order to solve this problem, the microneedle therapy emerged as the times require. In terms of microneedle therapy, the skin soft tissue is punctured by a micro needle-like apparatus to form minimally invasive stimulation, microchannels are constructed in the skin tissue, while the drugs or effective constituents are administered synchronously or asynchronously, so as to achieve the medical technology for treatment or beauty treatment. The drug administration methods include smearing the drugs on the skin or applying drug solution to the microneedles.

This method has apparently higher administration efficiency than smearing, but the skin needs to be pierced repeatedly by the microneedle roller, so that enough drugs can enter into the skin. Although the microneedles are very fine, the soft tissues are still mechanically injured to some extent in the piercing process of microneedles, thereby often causes a severe pain. Anaesthesia is often required, and there is certain bleeding.

### SUMMARY OF THE INVENTION

The purpose of the present invention is to provide a micro frozen body for loading drugs and the loading device thereof.

In the present invention, the liquid medicine or liquid that can be absorbed by the human body is frozen and solidified at low temperature to form a solid micro-frozen body or by adsorbing the drug on the micro-frozen body, so that the drug is loaded on the micro-frozen body. After the micro-frozen body enters the tissue The release of the drug within a certain period of time solves the problem of low loading efficiency of the microneedle drug solution.

To this end, the invention provides a drug-loaded micro-frozen body, the micro-frozen body contains a drug, and the micro-frozen body enters the human body to release the drug to the target group tissue.

More particularly, wherein the micro frozen body is formed of frozen liquid, the micro-frozen body enters the human body and melts at body temperature to release drugs to the target group tissue, the drug loading methods of the micro frozen body include:
(1) drugs loaded in micro frozen body;
(2) the overall micro frozen body is formed by freezing the liquid mixed with drugs;
(3) the overall micro frozen body is formed of frozen liquid drug.

More particularly, wherein the micro frozen body is formed of frozen liquid, said drug is adsorbed on the surface of micro frozen body.

More particularly, wherein the micro frozen body includes a needle body for puncture formed of frozen liquid, the length of the needle body is 0.5˜10 mm.

More particularly, wherein the micro frozen body contains active constituents for repair or treatment, the liquid includes stem cell supernatant.

More particularly, wherein the stem cell supernatant includes exosome and/or growth factor and/or cytokine.

More particularly, wherein the micro frozen body is formed by cryogenic freezing at −18° C.˜−80° C., the drug release time of the micro frozen body in the organism is 1 minute to 10 days.

More particularly, wherein the fission is formed by freezing the liquid, the formed fission is frozen many times with liquids with different melting speeds at body temperature to form the micro frozen body or the formed fission is frozen many times with liquid to form a micro frozen body with a predetermined melting order, different fissions contain different drugs or the same drug, the micro frozen body releases the drugs contained in different fissions according to the predetermined order in human body.

A loading device for the micro frozen body for loading drugs stated in Claim 1, wherein the micro frozen body is disposed on a holder, the holder is removably installed in the base of storage device, the base is provided with a removable cover, the base is applicable to removable connection to the hand-held part of loading device.

More particularly, wherein the base is provided with a guide slot, the guide slot is removably connected to a guide plate, the guide plate makes the cover move outwards along the guide plate when the cover.

### BENEFITS OF THE PRESENT INVENTION

(1) The present invention uses cryogenic freezing to solidify liquid to form a solid-state micro frozen body, the micro freezing loaded drug or the micro frozen body itself is formed of solidified drug solution;
(2) The micro frozen body can be made into needles with certain hardness, the micro frozen body implements puncture, the low temperature of micro frozen body can reduce the aching feeling in the piercing process and constrict the capillary vessels on the site of puncture to reduce bleeding;
(3) In the embodiments of the present invention, the liquid includes the stem cell supernatant, there will be no scars left on the skin after puncture treatment.

### BRIEF DESCRIPTION OF THE DRAWINGS

FIG. 1 is a schematic diagram of micro frozen body and the holder thereof;

FIG. 2 is a schematic diagram of the loading device;

FIG. 3 is a schematic diagram of the loading device in another embodiment;

FIG. 4 is a schematic diagram of loading the micro frozen body in the hand-held device in FIG. 3;

FIG. 5 is an upward view of base and guide plate in FIG. 3;

FIG. 6 is a top view of needle body.

### DETAILED DESCRIPTION OF THE INVENTION

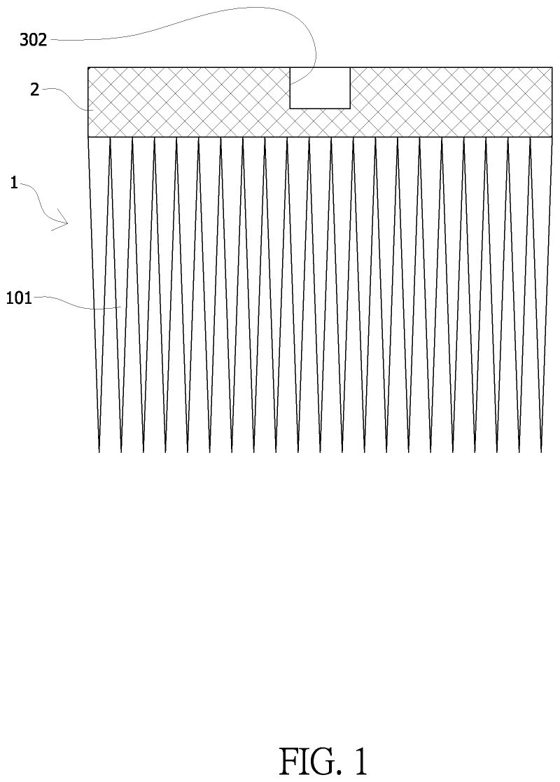

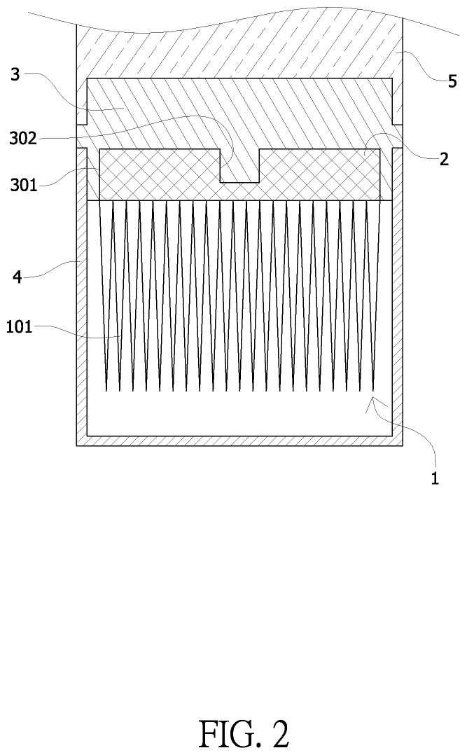

FIG. 1 and FIG. 2 show the micro frozen body for loading drugs of the present invention, the micro frozen body 1 is formed of frozen liquid, the drug loaded in the micro frozen body 1 or the overall micro frozen body 1 is formed by freezing drug solution, the micro frozen body 1 can melt and release the drug within a certain time in the organism. In this case, the drug can be dissolved in the liquid to form a drug solution or a liquid drug, which is frozen into a micro frozen body 1. The second way is to freeze the liquid to form a solid with a slot or a chamber, the drug is loaded in the chamber or slot of the solid, and refrozen to form a drug loaded micro frozen body 1. The third way is to use water as the liquid, the liquid is frozen to form a micro frozen body, when treatment is required, freeze-dried powder or atomized drug solution is sprayed over the micro frozen body, the freeze-dried powder and atomized drug solution are adsorbed on the surface of micro frozen body, implementing external loading of drug. The micro frozen body I is injected into the skin soft tissue, the micro frozen body 1 absorbs the heat in vivo and melts, the drug is released to the biological tissue in a specific position, in comparison to traditional adsorption of loaded drug, the drug loaded in the micro frozen body 1 can be completely released, and easily absorbed by the organism.

In the above case, the micro frozen body is formed at −18° C.˜−80° C., the optimal freezing temperature is −80° C.

In the above case, different therapeutic or repair constituents are selected according to the treated or repaired indications, e.g. repair solution or effective constituents or nutrient constituents, the carrier can be selected according to the therapeutic or repair constituents. In this case, active constituent can be used for skin repair and treatment, the carrier is said liquid, including stem cell supernatant, the stem cell supernatant can be used as the carrier of other active constituents, e.g. active proteins as polypeptide and oligopeptide; and it contains active constituents which promote cell growth and repair the damaged cells, so the stem cell supernatant can treat and repair minimal wound tissues, and the wounded skin is free of scars. Furthermore, the stem cell supernatant can include one or multiple kinds of exosome, growth factor and cytokine.

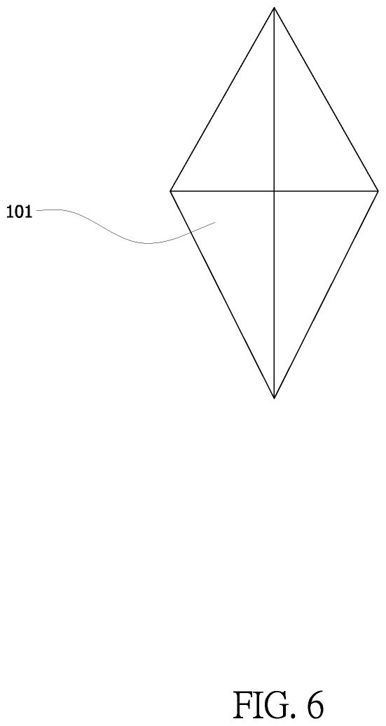

In the above case, as shown in FIG. 6, the micro frozen body 1 includes a needle body 101 formed of frozen liquid for puncture, according to the repaired and treated indications, the length of needle body 101 can be 0.5-10 mm, the cross section of needle body 101 is triangular, rhombic or circular, or in other shapes of the existing technology.

In the above case, different drugs are selected according to the indications, and micro frozen bodies with different slow release effects and needle bodies in different lengths can be selected for skin soft tissues of different depths, multiple embodiments are given below:
Embodiment 1: the indication is chloasma, the length of needle body is 0.5 mm.
Embodiment 2: the indication is hair growth, the length of needle body is 1 mm-4 mm, the optimal length is 2 mm.
Embodiment 3: the indication is acne scars, the length of needle body is 1 mm-3 mm, the optimal length is 1.5 mm.
Embodiment 4: wound repair, the length of needle body is 1 mm-10 mm, the wounds include operative wound and traumatic wound surface.

In the above case, in the fabrication of micro frozen body with a mold, as the micro frozen body is brittle and adhesive to some extent, demolding is difficult to some extent. In this case, an absorbable material is used as the carrier of micro frozen body, e.g. polyglycolide material or the absorbable material mentioned in CN103933620B patent document. The absorbable material is made into a thin-walled carrier with a chamber, the carrier is placed in a mold matching the carrier, a liquid is injected into the carrier, and frozen to form a micro frozen body, both of the carrier and micro frozen body are unloaded from the mold. The melting speed of micro frozen body can be adjusted to some extent by the contact surface area, shape and structure (e.g. hollow) of needle body and the thermal conductivity of micro frozen bodies formed of different liquids, by using said method, for example, the absorbable carrier of micro frozen body is divided into multiple sections, each fission can be separated by the interlayer made of absorbable carrier, each section is frozen into a fission, each fission has a different drug release rate, implementing differential sustained medication time of different drugs. Additionally, the fission is frozen by using said method, the formed fission is provided with different liquids and frozen, different fissions have different melting speeds or drug release rates at body temperature, a micro frozen body with multiple fissions is formed after multiple times of freezing, or the formed fission is frozen many times with liquid to form a micro frozen body with a predetermined melting order, for example, by layered freezing, the fission inside the micro frozen body is formed by freezing, and then different liquids are frozen outside the fission to form an outer fission, said procedure is repeated, so that the micro frozen body has multiple fissions from inside to outside, the melting proceeds from outside to inside, so that the fissions melt in different orders.

Different fissions contain different drugs or the same drug, the micro frozen body releases the drugs contained in different fissions according to the predetermined order in the human body. Additionally, the drug to be released later is frozen at first and formed in the inner layer of micro frozen body, the drug to be released earlier is loaded in the outer layer of micro frozen body, so as to implement asynchronous administration. The drug release time is generally 1 minute to 10 days.

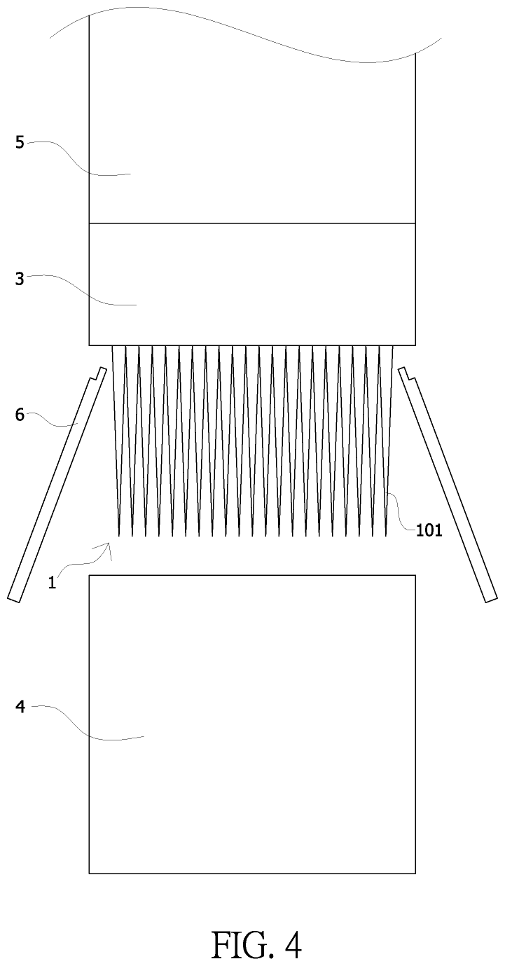

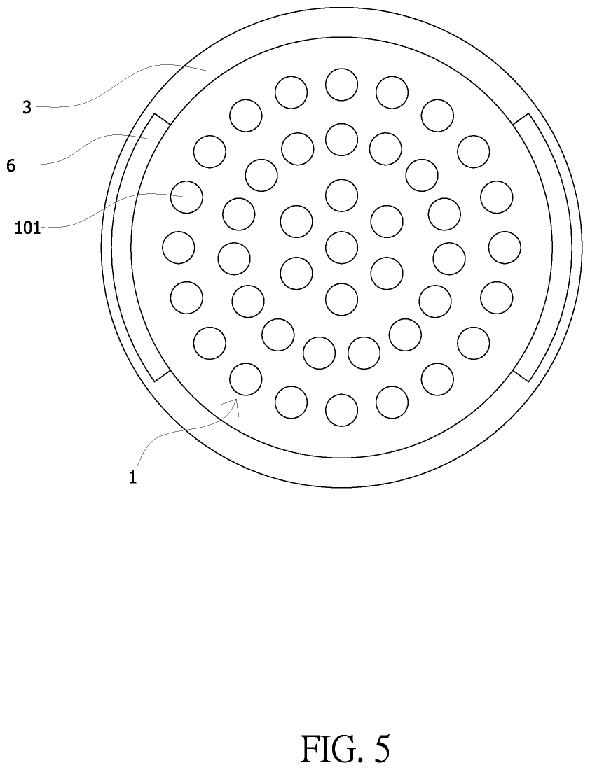

The present invention includes a loading device for said micro frozen body for loading drugs, as shown in FIG. 1 to FIG. 5, the micro frozen body 1 is disposed on a holder 2, the holder 2 is removably installed in a base 3 of storage device, the base 3 is provided with a removable cover 4, said base 3 is applicable to removable connection to the hand-held part 5 of loading device. In this case, the micro frozen body I includes a needle body 101, the needle body 101 and holder 2 are frozen together, the storage device includes a base 3 and a cover 4, the base 3 can be provided with a fixing slot 301, the holder 2 is matched with fixing slot 301, when the micro frozen body 1 is formed, the base 3 is clamped, the holder 2 and fixing slot 301 are tightened up or connected by buckle or thread. FIG. 2 discloses a screwing method, the base 3 is provided with a boss matched with the slot 302 on the holder 2. One end of base 3 is provided with an external thread, one end of hand-held part 5 is provided with an internal thread matched with the external thread of base 3. The other end of base 3 can be fixed to the thread of cover 4. When in use, the storage device is taken out by forceps, and the base 3 is fixed to the thread of hand-held part 5, the cover 4 of storage device is removed, the treatment region is aligned and punctured, the thawing time of micro frozen body 1 is often set as several minutes, keep pressing till the micro frozen body 1 is completely molten. When a slower release of micro frozen body 1 is required, the hand-held part 5 can be turned reversely while the base 3 is being pressed to disengage the hand-held part 5 from the base 3, the lower surface of holder 2 can be provided with adhesives, so as to be pasted on the skin surface. Certainly, the slot 302 can be disposed on the base 3, and the boss is disposed on the holder 2. The micro frozen body 1 is formed and loaded in the storage device, the storage device is stored and transported at −20° C., the storage device is connected to the hand-held device 5 before use.

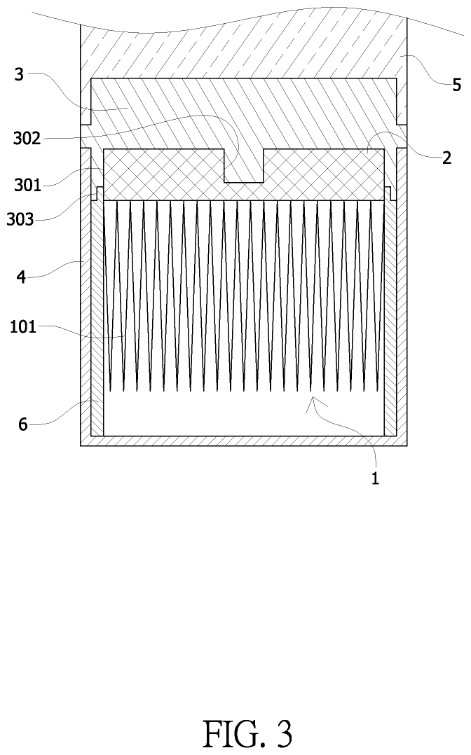

FIGS. 3 to 5 show another embodiment, differing from the above embodiment, the base 3 is provided with an insertion slot 303, the insertion slot 303 is removably connected to a guide plate 6, e.g. inserting or buckling, the guide plate 6 makes the cover 4 move outwards along the guide plate 6 when the cover 4 of storage device is being opened, so as to avoid contacting the micro frozen body 1 when the cover 4 is being opened to damage micro freezing. When in use, the hand-held part 5 is connected to the base 3, the cover 4 is screwed out along the guide plate 6, the guide plate 6 is pulled out or pulled out by pressing the buckle before press puncture.

We claim:

1. A micro frozen body for loading drugs, comprising drug, and entering a human body to release the drugs to a target group of tissue.

2. The micro frozen body defined in claim 1, wherein the micro frozen body is formed of frozen liquid, the micro-frozen body enters the human body and melts at body temperature to release drugs to the target group tissue, the drug loading methods of the micro frozen body include:
(1) drugs loaded in micro frozen body;
(2) the overall micro frozen body is formed by freezing the liquid mixed with drugs;
(3) the overall micro frozen body is formed of frozen liquid drug.

3. The micro frozen body defined in claim 1, wherein the micro frozen body is formed of frozen liquid, said drug is adsorbed on the surface of micro frozen body.

4. The micro frozen body defined in claim 1, wherein the micro frozen body includes a needle body for puncture formed of frozen liquid, the length of the needle body is 0.5-10 mm.

5. The micro frozen body defined in claim 1, wherein the micro frozen body contains active constituents for repair or treatment, the liquid includes stem cell supernatant.

6. The micro frozen body defined in claim 5, wherein the stem cell supernatant includes exosome and/or growth factor and/or cytokine.

7. The micro frozen body defined in claim 2, wherein the micro frozen body is formed by cryogenic freezing at −18° C.˜−80° C., the drug release time of the micro frozen body in the organism is 1 minute to 10 days.

8. The micro frozen body defined in claim 5, wherein the fission is formed by freezing the liquid, the formed fission is frozen many times with liquids with different melting speeds at body temperature to form the micro frozen body or the formed fission is frozen many times with liquid to form a micro frozen body with a predetermined melting order, different fissions contain different drugs or the same drug, the micro frozen body releases the drugs contained in different fissions according to the predetermined order in human body.

9. A loading device for the micro frozen body for loading drugs stated in claim 1, wherein the micro frozen body is disposed on a holder, the holder is removably installed in the base of storage device, the base is provided with a removable cover, the base is applicable to removable connection to the hand-held part of loading device.

10. The loading device defined in claim 9, wherein the base is provided with a guide slot, the guide slot is removably connected to a guide plate, the guide plate makes the cover move outwards along the guide plate when the cover.
